(19) United States
(12) Patent Application Publication
(10) Pub. No.: US 2005/0090849 A1
(43) Pub. Date: Apr. 28, 2005

(54) Angled tissue cutting instruments and method of fabricating angled tissue cutting instrument having flexible inner tubular members of tube and single wrap construction

(76) Inventor: Kenneth M. Adams, Jacksonville, FL (US)

Correspondence Address: Epstein & Gerken, Suite 340, 1901 Research Blvd, Rockville, MD, 20850, US

(21) Appl. No.: 10/689,628

(22) Filed: Oct. 22, 2003

(51) Int. Cl. A61B 17/32

(52) U.S. Cl. 606/180

(57) ABSTRACT

An angled tissue cutting instrument comprises an elongate angled outer tubular member and an elongate flexible inner tubular member rotatably disposed within the outer member to transmit torque to a cutting configuration of the inner member when the inner member is rotated relative to and within the outer member in forward and reverse rotational directions. The inner member comprises an inner tube, a helical cut formed along a length portion of the inner tube to impart flexibility and no more than a single layer of spiral wrap disposed over the helically cut length portion of the inner tube. A method of fabricating an angled tissue cutting instrument involves forming the helical cut in an inner tube having an inner diameter the same size as the inner diameter of the straight inner tubular member of a straight tissue cutting instrument of the same diametric size as the angled tissue cutting instrument.

### BACKGROUND OF THE INVENTION

1. Field of the Invention

The present invention relates generally to tissue cutting instruments having an elongate inner member rotatably disposed in an elongate outer tubular member to cut anatomical tissue and, more particularly, to angled tissue cutting instruments and methods of fabricating angled tissue cutting instruments having an elongate flexible inner tubular member rotatably disposed in an elongate angled outer tubular member.

2. Discussion of the Related Art

Surgical cutting instruments in which an elongate inner member is rotated within an elongate outer tubular member have become well accepted in surgical procedures where access to a cutting site in a patient's body is gained via a narrow or small size natural or surgically created anatomical opening or passage establishing communication with the cutting site from externally of the patient's body. Typically, the outer tubular member includes a distal end with an opening defining a cutting port or window, and the inner member includes a distal end carrying a cutting configuration exposed by or from the cutting port for engaging anatomical tissue at the cutting site. Proximal ends of the inner and outer members ordinarily include hubs which attach to a powered handpiece disposed externally of the patient's body and having a motor for rotating the inner member relative to and within the outer member. The cutting configuration of the inner member can have various configurations depending upon the surgical procedure to be performed, the type of tissue to be cut and/or the desired cutting action. The opening in the distal end of the outer member may be suitably configured to cooperate with the particular cutting configuration of the inner member to cut anatomical tissue. Often the inner member is tubular so that material, including loose tissue resulting from a cutting procedure, can be aspirated from the cutting site through the lumen of the inner member. Many tissue cutting instruments are designed to allow irrigating fluid to flow along the instruments for discharge at the cutting site, and some tissue cutting instruments are designed for flow of irrigating fluid between the outer and inner members. It is advantageous in tissue cutting instruments for the direction of rotation of the inner member to be reversible during operation for operation of the instruments in both forward and reverse rotational directions. An example of a rotary tissue cutting instrument of the aforementioned type is described in U.S. Pat. No. 4,203,444 to Bonnell et al for use in performing arthroscopic knee surgery.

Many tissue cutting instruments are straight, with longitudinally or axially straight inner and outer members as represented by the Bonnell et al patent. In straight tissue cutting instruments, it is typical for the outer tubular member to comprise an outer tube and for the inner tubular member to comprise an inner tube having an outer diameter for being accommodated in the inner diameter of the outer tube while allowing the inner tube to rotate within the outer tube. Typically, there is a small annular gap or clearance between the outer diameter of the inner tube and the inner diameter of the outer tube, and irrigating fluid may flow along this gap or clearance. Straight tissue cutting instruments are available in different diametric sizes corresponding to the outer diameter of the outer tube, and illustrative standard diametric sizes for straight tissue cutting instruments include 2.9 mm, 3.5 mm, 4.0 mm and 4.5 mm. Usually, it is desirable to minimize the outer diameter of the outer member to minimize the size of the anatomical opening or passage through which the instrument may be inserted in order to position the cutting configuration at a cutting site in a patient's body, to facilitate maneuverability of the instruments, and to enhance visibility of the cutting site. While design factors limit the extent to which the wall thicknesses of the inner and outer tubes may be minimized, it is beneficial to maximize the inner diameter of the inner tube in relation to the outer diameter of the outer tube for greater aspiration efficiency and lower risk of tissue clogging in the lumen of the inner tube. Clogging or jamming of tissue cutting instruments due to tissue build-up undesirably leads to the need for frequent cleaning or substitution of the instruments during use, which is time consuming and increases the duration of the surgical procedure to the detriment of the patient and the surgeon.

In many surgical procedures, it is advantageous for the tissue cutting instruments to be non-straight or angled to access cutting sites which are not accessible or are more difficult to access with straight cutting instruments. Angled tissue cutting instruments normally comprise an elongate angled outer tubular member and an elongate flexible inner tubular member which conforms to the angled configuration of the outer member while being rotatable therein. The angled configuration of the outer member can be formed by various angles, bends or curves, as limited by the ability of the flexible inner tubular member to bend. The angled outer tubular member typically comprises an angled outer tube, and angled tissue cutting instruments are typically available in different sizes corresponding to the outer diameter of the angled outer tube. Angled tissue cutting instruments are commonly available in the same diametric sizes as straight tissue cutting instruments, and representative standard diametric sizes for angled tissue cutting instruments include 2.9 mm, 3.5 mm, 4.0 mm and 4.5 mm. As with straight tissue cutting instruments, it is desirable to minimize the outer diameter of the angled outer member and to maximize the inner diameter of the flexible inner member in relation to the outer diameter of the outer member while retaining adequate design strength and functionality.

The flexible inner members of many angled tissue cutting instruments utilize spirally or helically wound coils or springs to transmit torque to rotate the cutting configuration when the inner members are rotated within the outer members. Flexible inner members that employ a single spirally or helically wound coil to impart flexibility while transmitting torque are represented by U.S. Pat. No. 4,466,429 to Loscher et al and No. 4,445,509 to Auth. A single coil tends to unwind when rotated in a direction opposite its winding so that torque can only be transmitted efficiently in one rotational direction. Accordingly, angled tissue cutting instruments utilizing this type of flexible inner member cannot be operated in both forward and reverse rotational directions.

Flexible inner tubular members having a plurality of coaxial spirally or helically wound coils disposed one on top of the other and wound in alternating opposite directions relative to one another have been used in angled tissue cutting instruments to transmit torque in both rotational directions. U.S. Pat. No. 4,646,738 to Troft describes an angled tissue cutting instrument in which the flexible inner tubular member comprises separate distal and proximal end portions and a composite spiral interposed between the distal and proximal end portions to allow the inner tubular member to bend. The composite spiral is similar to the flexible shaft disclosed in U.S. Pat. No. 177,490 to Fones et al and is made up of an inner spiral, a middle spiral and an outer spiral arranged one on top of the other with their windings alternating in direction. The distal and proximal end portions include reduced diameter neck portions which are telescopically received within opposite ends of the inner spiral to facilitate welding of the distal and proximal end portions to opposite ends of the composite spiral. Each spiral adds thickness to the overall wall thickness of the composite spiral and, therefore, to the overall wall thickness of the flexible inner tubular member. Consequently, the flexible inner tubular member of the Trott instrument will have a smaller inner diameter for a particular size angled outer tubular member than the inner diameter of the inner tubular member of a straight tissue cutting instrument of the same size. The angled tissue cutting instrument disclosed by Trott would thusly have inferior aspiration efficiency and a greater risk of clogging than a counterpart straight tissue cutting instrument of the same diametric size. Another disadvantage of the flexible inner tubular member used in the Trott instrument is that the neck portions tend to stiffen the composite spiral in the vicinity of the cutting tip thereby preventing the inner member from bending adjacent the cutting tip. In addition, it is possible for the separate components to become detached from one another during use such that torque can no longer be effectively transmitted to the cutting configuration. Angled tissue cutting instruments in which the flexible inner tubular member is like that disclosed in the Trott patent are described in U.S. Pat. No. 5,286,253 to Fucci and No. 5,529,580 to Kusunoki et al.

U.S. Pat. No. 5,314,438 to Shturman and U.S. Pat. No. 6,217,595 to Shturman et al relate to a flexible drive shaft comprising inner and outer oppositely wound helical wire layers along the entire length of the drive shaft. The drive shaft of the Shturman patent is referred to in the Shturman et al patent as being difficult and time-consuming to manufacture. The drive shaft of the Shturman et al patent has its outer helical layer made up of a single wire and its inner helical layer made up of a plurality of wires, which must all be wound around a forming mandrel so that the drive shaft requires many parts and is still difficult and time-consuming to manufacture. In the flexible drive shafts of the Shturman and Shturman et al patents, the inner helical layers need only be large enough to accommodate a guide wire, and maximizing the inner diameter of the inner helical layers is not an issue. Flexible shafts or tubular members comprising two layers of helical windings or coils have many of the same disadvantages as flexible tubular members that have three helical windings or coils.

Another disadvantage associated with the use of helical coils or springs to transmit torque while imparting flexibility is the tendency of the coils or springs to require tightening or preloading. Furthermore, coils or springs have a tendency under certain loading conditions to relax or unwind, and thus expand, thereby increasing the possibility of the inner member binding within the outer member. Coils or springs may undesirably have sizable gaps or spaces between the coils, especially on bending, and relaxation may increase the size of the gaps or spaces.

Another approach to flexible inner tubular members of angled tissue cutting instruments has involved forming relief apertures or slots in solid inner tubes to impart flexibility to the inner tubes as represented by U.S. Pat. No. 5,152,744 and No. 5,322,505 to Krause et al. In the angled tissue cutting instruments described in the aforementioned Krause et al patents, the inner tubes have discrete, unconnected apertures or slots formed therein such that torque transmission is limited.

U.S. Pat. No. 5,807,241 to Heimberger discloses a flexible tube particularly useful as a shank for a flexible endoscope. The flexible tube is formed by cutting a gap in a closed path in a longitudinally straight solid tube to form interlocking but completely materially or physically separated tube sections that allow the tube to bend axially. The flexible tube may not be well suited for use as a rotatable inner tubular member of a surgical cutting instrument since its torque capabilities may be limited to relatively low single direction and bidirectional rotational speeds. Also, it is possible for the individual tube sections to disconnect or become detached when the tube is bent.

Angled tissue cutting instruments having inner tubes with continuous helical cuts therein to impart flexibility are illustrated by U.S. Pat. No. 6,053,922 to Krause et al, No. 6,312,438 B1 to Adams and No. 6,533,749 B1 to Mitusina et al. In the angled tissue cutting instruments disclosed by Krause et al '922, no additional layer of material is secured over the helically cut inner tube. Accordingly, the instrument may be suitable for transmitting torque in one direction only and may be of limited torsional strength. The angled tissue cutting instruments described in the Adams and Mitusina et al patents have flexible inner tubular members including flexible regions formed by a helical cut in an inner tube and two spiral wrap layers disposed over the helical cut in the inner tube one on top of the other in alternating directions. The instruments disclosed in the Adams and Mitusina et al patents overcome the disadvantages of wound helical coils or springs and can effectively transmit torque in both rotational directions at relatively high rotational speeds with minimal wind-up and with the structurally interconnected inner tube eliminating the problems of disconnection or detachment of the inner tube. Moreover, the use of a helically cut inner tube achieves a high degree of bendability and allows flexibility to be imparted to the inner tube adjacent the cutting configuration. However, each spiral wrap layer adds thickness to the overall wall thickness of the flexible inner tubular members such that the flexible inner tubular member for a particular size angled outer tubular member has a smaller inner diameter than the straight inner tubular member for the same size straight outer tubular member. For example, a 4 mm angled tissue cutting instrument will typically be fabricated utilizing an inner tube of the same inner diametric size as the inner tube of a 3.5 mm straight tissue cutting instrument, and a 3.5 mm angled tissue cutting instrument will typically be fabricated using an inner tube of the same inner diametric size as the inner tube of a 2.9 mm straight cutting instrument. In addition, each spiral wrap layer adds material and labor costs to the angled tissue cutting instrument.

In order to increase aspiration efficiency, reduce the risk of clogging, and lower the cost of angled tissue cutting instruments, it would be desirable to increase the inner diameters of the flexible inner tubular members of angled tissue cutting instruments for various diametric sizes of angled outer members, to allow straight and angled tissue cutting instruments of the same diametric size to have the same or essentially the same aspiration efficiencies and risks of clogging, to allow the inner tubular members of straight and angled tissue cutting instruments of the same diametric size to be fabricated using inner tubes of the same inner diametric size, and to reduce the labor and materials needed to fabricate the flexible inner tubular members of angled tissue cutting instruments while retaining the benefits of a helically cut inner tube and providing minimal wind-up, effective bidirectional torque transmission for angles of various magnitudes, radii of curvature and directions, and sufficient design strength and functionality.

### SUMMARY OF THE INVENTION

Accordingly, it is a primary object of the present invention to overcome the aforementioned disadvantages of prior angled tissue cutting instruments and prior flexible inner tubular members of angled tissue cutting instruments.

Another object of the present invention is to increase the aspiration efficiency through the flexible inner tubular members of angled tissue cutting instruments.

A further object of the present invention is to decrease the risk of clogging in angled tissue cutting instruments.

An additional object of the present invention is to lower the cost of flexible inner tubular members of angled tissue cutting instruments.

It is also an object of the present invention to reduce the labor and materials needed to fabricate the flexible inner tubular members of angled tissue cutting instruments.

The present invention also has as an object to increase the inner diameters of the flexible inner tubular members of angled tissue cutting instruments in relation to the outer diameters of the angled outer tubular members rotatably receiving the flexible inner tubular members.

The present invention has as another object to reduce the overall wall thicknesses of the flexible regions of flexible inner tubular members of angled tissue cutting instruments for various outer diametric sizes of angled outer tubular members rotatably receiving the flexible inner tubular members with minimal clearance between the inner and outer tubular members.

A still further object of the present invention is to allow straight and angled tissue cutting instruments of the same diametric size to have the same or essentially the same aspiration efficiency and risk of clogging.

Moreover, it is an object of the present invention to permit the flexible inner tubular member of an angled tissue cutting instrument to be fabricated using an inner tube having the same inner diameter as the inner tube of a straight tissue cutting instrument of the same diametric size as the angled tissue cutting instrument.

Still a further object of the present invention is to increase the inner diameters of the flexible inner tubular members of various standard diametric sizes of angled tissue cutting instruments while retaining the benefits of constructing the flexible inner tubular members from helically cut inner tubes for torque transmission in forward and reverse rotational directions.

The aforesaid objects are achieved individually and in combination, and it is not intended that the present invention be construed as requiring two or more of the objects to be combined unless expressly required by the claims attached hereto.

Some of the advantages of the present invention are that the cutting configuration of the flexible inner tubular member may be a cutting tip formed integrally, unitarily with the inner tube or as a separate component attached to a forward end of the inner tube; various different cutting configurations can be used including end cutters, side cutters, trimmers, resectors, shavers, abraders and burs; the cutting configuration can be configured to produce various cutting actions independently or in cooperation with the distal end of the outer tubular member including side cutting, end cutting, trimming, burring, abrading and resection; the inner tubular member can be angled or bent adjacent the cutting configuration; the outer tubular member can include one or a plurality of angles at various locations along the length of the outer tubular member; the angle or angles in the outer tubular member may be formed by various curves, bends or angles of various magnitudes and radii of curvature and may extend in various directions; the flexible inner tubular member may have one or a plurality of flexible regions; plural flexible regions may be spaced longitudinally from one another along the length of the inner tube for being disposed in the angles, respectively, of the outer tubular member; a single flexible region may be of sufficient length to be disposed in more than one angle of the outer tubular member; irrigating fluid can be supplied along the angled tissue cutting instrument for discharge at a cutting site; irrigating fluid can be supplied along the angled tissue cutting instrument internally and/or externally; irrigating fluid can be supplied between the inner and outer tubular members; the inner tubular member can have one or more aspiration ports at various locations for aspirating materials at the cutting site into the lumen of the inner tubular member; the angled tissue cutting instrument can be driven by any suitable powered surgical handpiece capable of rotating the inner tubular member relative to and within the outer tubular member; and the angled tissue cutting instrument is useful in various types of surgery including surgery of the head and neck as well as other types of surgery.

These and other objects, advantages and benefits are realized with the present invention as generally characterized in an angled tissue cutting instrument comprising an elongate angled outer tubular member and an elongate flexible inner tubular member rotatably disposed within the outer tubular member to transmit torque in forward and reverse rotational directions. The outer tubular member includes a proximal end, a distal end, a bend between the proximal end and the distal end, and an opening at the distal end defining a cutting port in communication with the lumen of the outer tubular member. The inner tubular member comprises a proximal end, a distal end, an elongate inner tube between the proximal end and the distal end of the inner tubular member, and a cutting configuration at the distal end of the inner tubular member for exposure by the cutting port to cut anatomical tissue when the inner tubular member is rotated within the outer tubular member. A continuous helical cut is formed along a length portion of the inner tube, which is of solid wall construction prior to the helical cut being formed therein. The helical cut is formed in the inner tube at an angle in a first direction about the tube to impart flexibility along the length portion by which the inner tubular member conforms to the angled outer tubular member while being rotated within the angled outer tubular member. No more than a single layer of spiral wrap is secured over the inner tube, the spiral wrap extending along the length portion at the same angle as the helical cut but in a second direction, opposite the first direction, about the inner tube. A flexible region of the inner tubular member is formed by the helically cut length portion of the inner tube and the single layer of spiral wrap disposed over the helically cut length portion. The flexible region is in correspondence with the bend in the angled outer tubular member such that the flexible region is disposed within and conforms to the bend while transmitting torque to the cutting configuration when the inner tubular member is rotated relative to and within the outer tubular member in the forward and reverse rotational directions. The lumen of the inner tube defines an aspiration passage through the flexible inner tubular member, and an aspiration port at the distal end of the inner tubular member establishes communication with the aspiration passage. Because the inner tubular member comprises the inner tube and no more than a single layer of spiral wrap disposed over the inner tube, the inner tube of a particular diametric size angled tissue cutting instrument has an inner diameter the same size as the inner diameter of the inner tubular member of a straight tissue cutting instrument of the same diametric size as the angled tissue cutting instrument. Accordingly, the angled tissue cutting instrument has the same or essentially the same aspiration efficiency and risk of clogging as the straight tissue cutting instrument of the same diametric size. Also, the angled tissue cutting instrument and, in particular, the flexible inner tubular member thereof, can be fabricated at lower cost than the flexible inner tubular members of prior angled tissue cutting instruments.

The helical cut may be formed in the inner tube in a stepped pattern comprising repeating interconnected steps. Each step comprises a transverse cut segment extending transverse to the length of the inner tube at the angle in the first direction and a longitudinal cut segment extending from the transverse cut segment along the length of the inner tube. The transverse cut segment meets the longitudinal cut segment at an outside corner forming a step configuration. The longitudinal cut segment extends from the transverse cut segment at the outside corner to an inside corner at which the longitudinal cut segment meets the transverse cut segment of the next step. The steps repeat at about 100 degree rotational intervals about a central longitudinal axis of the inner tube. In a preferred embodiment, the angle of the helical cut, which is also the angle of the spiral wrap in the opposite direction, is 20 degrees. The helical cut tightens as the inner tubular member is rotated relative to and within the outer tubular member in a forward rotational direction. The spiral wrap layer tightens down onto the inner tube when the inner tubular member is rotated in a reverse rotational direction such that the inner tubular member transmits torque to the cutting configuration in both forward and reverse rotational directions.

The present invention is further characterized in a method of fabricating an angled tissue cutting instrument and, in particular, the flexible inner tubular member of an angled tissue cutting instrument. The method involves forming a continuous helical cut along a length portion of an elongate inner tube at an angle in a first direction about the tube to impart flexibility along the length portion, the inner tube being of solid wall construction prior to having the helical cut formed therein and having an inner diameter the same size as the inner diameter of an elongate inner tube forming the inner tubular member of a straight tissue cutting instrument of the same diametric size as the angled tissue cutting instrument. A continuous strip of material is wrapped spirally over the helically cut length portion of the inner tube in a second direction, opposite the first direction, and at the same angle as the helical cut to form no more than a single layer of spiral wrap over the inner tube. Opposing ends of the strip of material are secured to the inner tube to form a flexible inner tubular member having a flexible region along the length portion of the inner tube. The flexible inner tubular member is inserted for rotation within an angled outer tubular member with the flexible region disposed within a bend in the outer tubular member and a cutting configuration of the flexible inner tubular member exposed by a cutting port in a distal end of the outer tubular member, with the outer tubular member having an outer diameter the same size as the outer diameter of a straight outer tubular member of the straight tissue cutting instrument of the same diametric size as the angled tissue cutting instrument. The helical cut may be formed in an inner tube which is the same as the inner tube used as the inner tubular member of the straight tissue cutting instrument of the same diametric size as the angled tissue cutting instrument. The helical cut may be formed in a stepped pattern. The helical cut may be formed in the inner tubes of 2.9 mm, 3.5 mm and 4.0 mm angled tissue cutting instruments, for example, using an inner tube having the same inner diameter as the inner tubes of 2.9 mm, 3.5 mm and 4.0 mm straight tissue cutting instruments, respectively.

Other objects and advantages of the present invention will become apparent from the following description of a preferred embodiment taken in conjunction with the accompanying drawings, wherein like parts in each of the several figures are identified by the same reference numerals.

### BRIEF DESCRIPTION OF THE DRAWINGS

FIG. 1 is a broken side view of a prior art tissue cutting instrument that is longitudinally or axially straight.

FIG. 2 is an exploded broken side view of the straight tissue cutting instrument of FIG. 1.

FIG. 3 is an exploded broken side view of a prior art angled tissue cutting instrument of the same size as the straight tissue cutting instrument of FIGS. 1 and 2.

FIG. 4 is an enlarged broken side view of a flexible region of the flexible inner tubular member of the angled tissue cutting instrument of FIG. 3.

FIG. 5 is an exploded broken side view of an angled tissue cutting instrument according to the present invention of the same size as the angled tissue cutting instrument of FIG. 3.

FIG. 6 is a broken side view of an inner tube of the flexible inner tubular member of the angled tissue cutting instrument of the present invention.

FIG. 7 is an enlarged broken side sectional view of a cutting configuration of the flexible inner tubular member.

FIG. 8 is a broken side view of the inner tube with a helical cut formed therein.

FIG. 9 is an enlarged detail view depicting a stepped pattern for the helical cut in the inner tube.

FIG. 10 is a broken side view of the inner tube with a single spiral wrap layer disposed over the helical cut to form the flexible inner tubular member.

### DETAILED DESCRIPTION OF THE PREFERRED EMBODIMENT

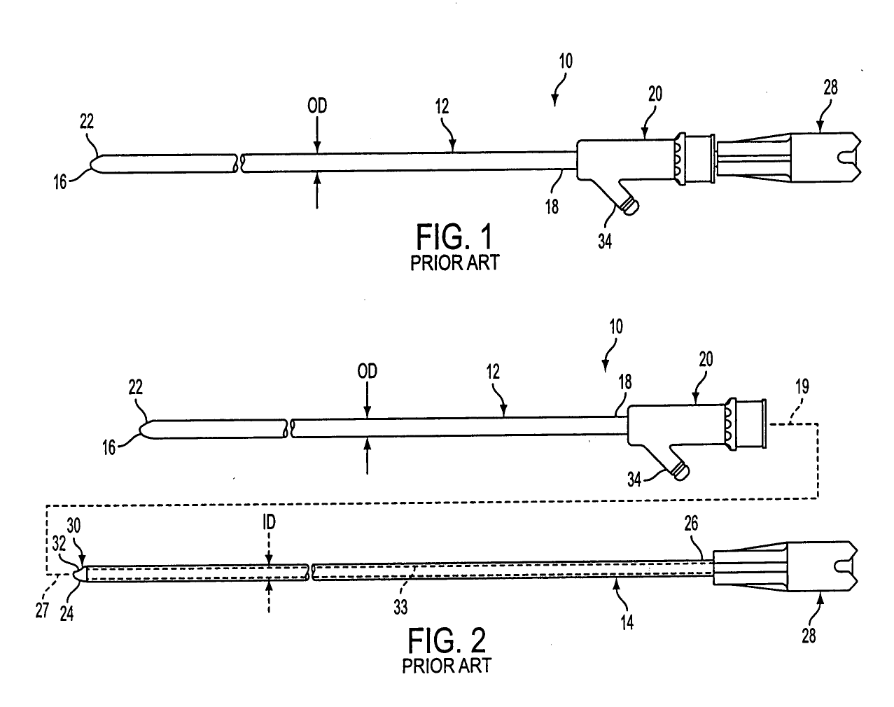

A prior art tissue cutting instrument 10 that is longitudinally or axially straight is illustrated in FIGS. 1 and 2. Straight tissue cutting instrument 10 comprises a longitudinally or axially straight elongate outer tubular member 12 and a longitudinally or axially straight elongate inner tubular member 14 rotatably disposed in outer tubular member 12. The outer tubular member 12 is typically made of stainless steel and includes a distal end 16, a proximal end 18 and a central longitudinal axis 19 that follows a straight line path. The proximal end 18 is typically attached to an outer member hub 20, which may be made of plastic. An opening is formed in the distal end 16 and defines a cutting port or window 22 providing communication with the lumen of the outer tubular member 12 from externally of distal end 16. The cutting port 22 can have various configurations and may be circumscribed by a peripheral edge.

The inner tubular member 14 is typically made of stainless steel and includes a distal end 24, a proximal end 26 and a central longitudinal axis 27 that follows a straight line path. The proximal end 26 is typically attached to an inner member hub 28, which may be made of plastic. The distal end 24 carries a cutting configuration 30, which may have various configurations to cut anatomical tissue. An opening is formed through the distal end 24 defining an aspiration or suction port 32 communicating with an aspiration or suction passage defined by the lumen 33 of the inner tubular member 14. The aspiration port 32 may have various configurations and may be disposed at various locations on the inner tubular member 14. The cutting configuration 30 may comprise a cutting edge circumscribing the aspiration port 32.

The outer and inner member hubs 20 and 28 are ordinarily coupled with a powered surgical handpiece (not shown) for rotating the inner tubular member 14 relative to and within the outer tubular member 12. The powered surgical handpiece maintains the longitudinal position of the outer and inner members 12 and 14 relative to one another so that the cutting configuration 30 is exposed by or from the cutting port 22 to cut anatomical tissue as the inner tubular member 14 is rotated within the outer tubular member 12. The peripheral edge of the outer tubular member that circumscribes the cutting port 22 may be configured to cooperate with the cutting configuration 30 to cut anatomical tissue. A representative powered surgical handpiece is disclosed in U.S. Pat. No. 5,916,231 to Bays, the entire disclosure of which is incorporated herein by reference.

In order to access anatomical tissue in a cutting procedure, the instrument 10 is typically introduced through a natural or surgically created anatomical opening or passage in a patient's body to position the distal end 16 of the outer tubular member 12 at a cutting site in the patient's body while the handpiece is maintained externally of the patient's body. Exposure of the cutting configuration 30 by or from the cutting port 22 allows anatomical tissue at the cutting site to be accessed and cut by the cutting configuration. The aspiration port 32 establishes communication between the cutting site and the lumen or aspiration passage 33 of the inner tubular member 14 and, when suction is produced in the lumen of the inner tubular member via the handpiece, materials such as tissue debris are drawn into the lumen of the inner tubular member via the aspiration port for aspiration from the patient's body. Where the cutting configuration 30 comprises a cutting edge circumscribing the aspiration port 32, the cutting edge and aspiration port register with the cutting port 22 as the inner member 14 rotates within the outer member 12. Irrigating fluid may be supplied externally or internally along the instrument 10 for discharge at the cutting site. As an example, irrigating fluid may be supplied between the outer and inner tubular members 12 and 14 for discharge through the cutting port 22. The outer member hub 20 may have a connector 34 for being coupled with a source of irrigating fluid, with the connector 34 communicating with the lumen of the outer tubular member 12 to supply the irrigating fluid between the inner diameter of the outer tubular member 12 and the outer diameter of inner tubular member 14. U.S. Pat. No. 5,957,881 to Peters et al and the Irrigating Straight Blades of the Medtronic Xomed Power System Blades of Medtronic Xomed, Inc. are representative of the straight tissue cutting instrument 10 and are incorporated herein by reference.

The outer tubular member 12 typically has an outer diameter OD identifying or corresponding to the size of the instrument 10, and the instrument 10 may be made available in different diametric sizes corresponding to different outer diameters OD. Many manufacturers of straight tissue cutting instruments identify the sizes of the instruments by the outer diameters of the outer tubular members, and typical standard sizes of straight tissue cutting instruments include 2.9 mm, 3.5 mm and 4.0 mm. The outer diameter OD of the outer tubular member 12 is ordinarily minimized to minimize the size of the anatomical opening or passage through which the instrument 10 may be introduced. The inner diameter ID of the inner tubular member 14 defines the diametric size of the aspiration passage defined by the lumen 33 of the inner tubular member. Maximizing the diametric size of the aspiration passage 33 provides greater aspiration efficiency through the inner tubular member, reduces the risk of tissue clogging in the lumen of the inner tubular member, and lowers the risk that the instrument will jam during a cutting procedure. The outer diameter of the inner tubular member 14 is typically received in the inner diameter of the outer tubular member 12 with a close fit, i.e. with minimal gap or clearance between the outer diameter of the inner tubular member and the inner diameter of the outer tubular member, while still allowing rotation of the inner tubular member within the outer tubular member and the flow of irrigating fluid between the inner and outer members. Design factors such as strength and functionality limit the extent to which the wall thicknesses of the outer and inner tubular members 12 and 14 may be minimized. Accordingly, each size of tissue cutting instrument 10 is ordinarily associated with an inner tubular member 14 having a particular inner diameter ID, with the inner diameters ID of the inner tubular members 14 generally increasing in diametric size as the size of the instrument increases.

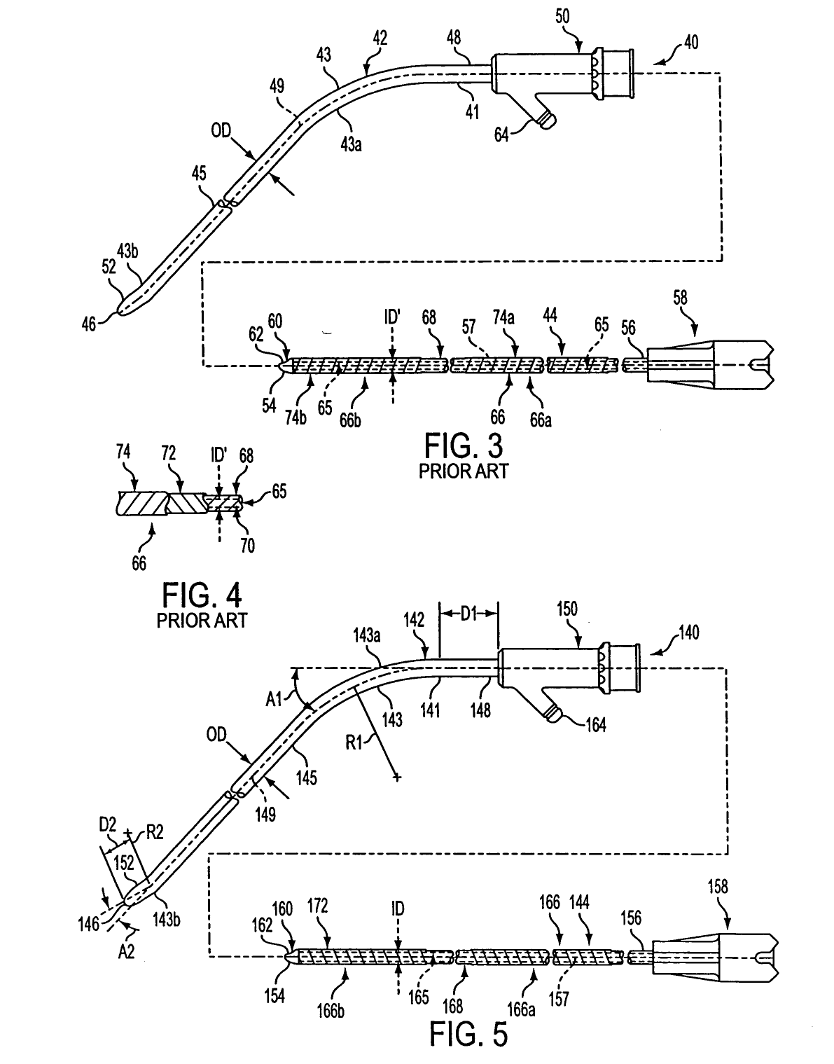

A prior art angled tissue cutting instrument 40 is depicted in FIG. 3 and comprises an angled elongate outer tubular member 42 and a flexible elongate inner tubular member 44 for being rotatably disposed in angled outer tubular member 42 as described above for straight tissue cutting instrument 10. The angled outer tubular member 42 includes a distal end 46, a proximal end 48 and a central longitudinal axis 49 that follows a non-straight or angled longitudinal path. The proximal end 48 is attached to outer member hub 50, and an opening is formed in the distal end 46 defining a cutting port or window 52 as described above for outer member 12. The outer member hub 50 has a connector 64 similar to the connector 34 described above for outer member 12. Angled outer tubular member 42 has one or more bends, curves or angles 43 which may be of various magnitudes and radii of curvature and may extend in various directions at various locations along the length of the outer tubular member 42. Angled outer tubular member 42 has a plurality of bends, curves or angles 43 of different magnitudes and different radii of curvature extending in different directions at longitudinally spaced locations along the length of the outer tubular member 42.

Angled outer tubular member 42 includes a straight proximal length portion 41 extending distally from outer member hub 50 to a proximal bend 43a, and a straight intermediate length portion 45 extending distally from proximal bend 43a to a distal bend 43b adjacent distal end 46. The central longitudinal axis 49 is contained in a plane, with the proximal and distal bends 43a and 43b extending in different directions within the plane. Looking at FIG. 3, the proximal bend 43a extends downwardly in this plane from the proximal length portion 41 and the distal bend 43b extends upwardly in this plane from the intermediate length portion 45. The proximal bend 43a defines an angle with the proximal length portion 41 that is different than the angle that the distal bend 43b defines with the intermediate length portion 45. The proximal bend 43a has a radius of curvature different than the radius of curvature of distal bend 43b.

The flexible inner tubular member 44 includes a distal end 54, a proximal end 56 and a central longitudinal axis 57 of variable configuration due to flexibility of the inner tubular member 44. The proximal end 56 is attached to an inner member hub 58, the distal end 54 carries a cutting configuration 60, and the inner tubular member 44 has an aspiration port 62 as described for inner tubular member 14. The flexible inner tubular member 44 has a lumen 65 defining an aspiration passage in communication with the aspiration port 62 as described above for inner tubular member 14. The flexible inner tubular member 44 has one or more flexible regions 66 for being disposed within the one or more bends 43 of the outer member 42 to allow the inner tubular member 44 to conform to the angled configuration of the angled outer tubular member 42 while being rotatable within the angled outer tubular member. The flexible inner tubular member 44 has two flexible regions, i.e. proximal flexible region 66a and distal flexible region 66b, each of sufficient length to extend within the proximal and distal bends 43a and 43b, respectively. The flexible regions 66a and 66b are longitudinally spaced from one another along the length of inner tubular member 44.

As shown in FIGS. 3 and 4, the flexible inner tubular member 44 comprises an elongate inner tube 68 having one or more continuous helical cuts 70 formed therein along the one or more flexible regions 66 to impart flexibility to the inner tube 68 along the one or more flexible regions 66. The distal end 54 may be formed integrally, unitarily or monolithically with the inner tube 68 or as a separate component attached to a forward end of the inner tube 68. The proximal end 56 may be defined by a rearward end of the inner tube 68. The lumen 65 of the inner tube 68 defines the aspiration passage of the flexible inner tubular member 44. The at least one helical cut 70 is formed through the wall thickness of the inner tube 68 and follows a helix angle in a first direction, i.e. clockwise (right-hand) or counterclockwise (left-hand), about the central longitudinal axis 57 of the inner tube 68. The inner tube 68 has a plurality of, namely two, continuous helical cuts 70 formed therein at longitudinally spaced locations along the length of the inner tube 68. The inner tube 68 has a proximal helical cut and a distal helical cut extending along the proximal and distal flexible regions 66a and 66b, respectively.

The flexible inner tubular member 44 further includes one or more inner or first spiral wraps forming one or more inner or first spiral wrap layers 72 disposed over the one or more helical cuts 70, and one or more outer or second spiral wraps forming one or more outer or second spiral wrap layers 74 disposed over the one or more inner spiral wrap layers 72. The inner tubular member 44 includes proximal and distal inner spiral wraps forming proximal and distal inner spiral wrap layers disposed over the proximal and distal helical cuts, respectively. Each inner spiral wrap layer 72 comprises a continuous strip of material wound over the helically cut inner tube 68 in a direction opposite the first direction of the helical cut and at an angle that is the same as the helix angle but in the opposite direction. Opposite ends of the strips of material forming the inner spiral wrap layers are secured to the tube 68 such as by welding. The flexible inner tubular member 44 includes proximal and distal outer spiral wrap layers 74a and 74b disposed over the proximal and distal inner spiral wrap layers, respectively. Each outer spiral wrap layer 74a and 74b comprises a continuous strip of material wound over the helically cut inner tube 68 in the same direction and at the same angle as the helical cut 70. Opposite ends of the strips of material forming the outer spiral wrap layers are secured to the inner tube 68 such as by welding.

Each flexible region 66 of the flexible inner tubular member 44 thusly comprises a continuous helical cut 70 in the inner tube 68, i.e. a helically cut length portion of the inner tube 68, an inner spiral wrap layer 72 disposed over the helically cut length portion and an outer spiral wrap layer 74 disposed over the inner spiral wrap layer 72. The proximal flexible region 66a is made up of a proximal helically cut length portion, a proximal inner spiral wrap layer and the proximal outer spiral wrap layer 74a. The distal flexible region 66b is made up of a distal helically cut length portion, a distal inner spiral wrap layer and the distal outer spiral wrap layer 74b. The proximal and distal flexible regions 66a and 66b are disposed in the proximal and distal bends 43a and 43b, respectively, of the outer tubular member 42 when the flexible inner tubular member 44 is rotatably disposed within the angled outer tubular member 42. The flexible regions 66a and 66b conform to the configurations of the bends 43a and 43b, respectively, as the inner member 44 is rotated relative to and within the outer member 42 via a powered surgical handpiece to transmit torque to the distal end 54, and the flexible inner tubular member 44 is capable of effectively transmitting torque when rotated in both forward and reverse rotational directions. Operation of angled tissue cutting instrument 40 to cut anatomical tissue in a cutting procedure is similar to that described above for straight tissue cutting instrument 10 except that the angled configuration of outer tubular member 42 may provide better access to some cutting sites than the straight outer tubular member 12. U.S. Pat. No. 6,312,438 B1 to Adams and U.S. Pat. No. 6,533,749 B1 to Mitusina et al disclose angled tissue cutting instruments that are representative of the angled tissue cutting instrument 40, and the disclosures of the Adams and Mitusina et al patents are incorporated herein by reference.

The outer tubular member 42 typically has an outer diameter OD corresponding to the size of the angled tissue cutting instrument 40, and the angled tissue cutting instrument 40 may be made available in different diametric sizes corresponding to different outer diameters OD. The angled tissue cutting instrument 40 may be made available in sizes corresponding to the sizes of straight tissue cutting instrument 10, and typical standard sizes of angled tissue cutting instruments include 2.9 mm, 3.5 mm and 4.0 mm. Oftentimes, angled and straight tissue cutting instruments having the same cutting configuration are made available as counterpart instruments of the same size. Accordingly, the outer diameter OD of straight outer tubular member 12 and the outer diameter OD of angled outer tubular member 42 will be the same size where the straight tissue cutting instrument 10 and the angled tissue cutting instrument 40 are the same size, and the angled outer tubular member 42 may be fabricated by bending the straight outer tubular member 12.

The inner diameter ID′ of the flexible inner tubular member 44 is defined by the inner diameter of the inner tube 68, and the overall wall thickness of the flexible inner tubular member 44 comprises the wall thickness of inner tube 68, the thickness of inner spiral wrap layer 72 and the thickness of outer spiral wrap layer 74. Each spiral wrap layer 72 and 74 is typically about 0.003 inch to about 0.006 inch in thickness. Since design factors limit the extent to which the wall thickness of outer tubular member 42, the wall thickness of inner tube 68 and the thicknesses of the strips of material forming the inner and outer spiral wrap layers 72 and 74 can be minimized, the flexible inner tubular member 44 will have an inner diameter ID′ that is smaller than the inner diameter ID of the straight inner tubular member 14 of the same size instrument in order for the inner tubular member 44 to be rotatably received in an angled outer tubular member 42 of the same outer diameter as the straight outer tubular member 12. For a particular size angled tissue cutting instrument 40, the inner tubular member 14 of a smaller size straight cutting instrument 10 is often used as the inner tube 68, such that the angled tissue cutting instrument has lower aspiration efficiency and greater risk of clogging and jamming than a straight tissue cutting instrument of the same size as the angled tissue cutting instrument. In addition to the inner and outer spiral wrap layers 72 and 74 adding wall thickness to the flexible inner tubular member 44, each spiral wrap layer 72 and 74 adds material and labor costs to the flexible inner tubular member 44, thereby making it more expensive to fabricate inner tubular member 44 as well as the instrument 40.

An angled tissue cutting instrument 140 according to the present invention is depicted in FIG. 5 and is similar to angled tissue cutting instrument 40 except for the construction of flexible inner tubular member 144. Angled tissue cutting instrument 140 includes an elongate angled outer tubular member 142 and an elongate flexible inner tubular member 144 for being rotatably disposed in outer tubular member 142. The angled outer tubular member 142 may be made of stainless steel and includes a distal end 146, a proximal end 148 and a central longitudinal axis 149 that follows a non-straight or angled longitudinal path. The proximal end 148 is attached to an outer member hub 150, which may be made of plastic, and an opening is formed in the distal end 146 defining a cutting port or window 152 in communication with the lumen of the outer tubular member as described above for straight outer tubular member 12. The outer member hub 150 has a connector 164, similar to connector 34, in communication with the lumen of the outer tubular member as also described above for outer tubular member 12. The angled outer tubular member 142 has one or more bends, curves or angles 143 of various magnitudes and radii of curvature extending in various directions at various locations along the length of the outer tubular member 142. Angled outer tubular member 142 has a plurality of bends, curves or angles 143 of different magnitudes and radii of curvature extending in different directions at longitudinally spaced locations along the length of the outer tubular member 142.

Angled outer tubular member 142 includes a straight proximal length portion 141 extending distally from outer member hub 150 to a proximal bend 143a, and a straight intermediate length portion 145 extending distally from proximal bend 143a to a distal bend 143b adjacent distal end 146. It should be appreciated that the outer tubular member 142 may be provided with only a single bend, and that the proximal bend 143a or the distal bend 143b may comprise the single bend. The central longitudinal axis 149 is contained in a plane, and the proximal and distal bends 143a and 143b extend in different directions within this plane. Looking at FIG. 5, the proximal bend 143a extends downwardly in this plane from the proximal length portion 141, and the distal bend 143b extends upwardly in this plane from the intermediate length portion 145. It should be appreciated that the one or more bends 143 can extend upwardly, downwardly or laterally such that the central longitudinal axis 149 does not have to be contained or lie in a plane. The proximal bend 143a defines an angle A1 with the central longitudinal axis of the proximal length portion 141 that is greater than the angle A2 that the distal bend 143b defines with the central longitudinal axis of the intermediate length portion 145. However, it should be appreciated that the proximal and distal bends 143a and 143b can define the same or different angles. The proximal bend 143a has a radius of curvature R1 greater than the radius of curvature R2 of distal bend 143b, but the proximal and distal bends 143a and 143b could have the same radius of curvature.

In one preferred embodiment, the proximal bend 143a defines an angle A1 with the proximal length portion 141 of about 45 degrees; the distal bend 143b defines an angle A2 with the intermediate length portion 145 of about 15 degrees; the proximal bend 143a begins a distance D1 of about 0.50 inch distally of the outer member hub 150; the proximal bend 143a has a radius of curvature R1 of about 1.50 inches; the distal bend 143b has a radius of curvature R2 of about 0.396 inch; and the radius of curvature R2 is located a distance D2 of about 0.25 inch from a distal end surface of the distal end 146.

The flexible inner tubular member 144 includes a distal end 154, a proximal end 156 and a central longitudinal axis 157 of variable configuration due to flexibility of the inner tubular member 144. The proximal end 156 is attached to an inner member hub 158, which may be made of plastic, the distal end 154 carries a cutting configuration 160, and the inner tubular member 144 has an aspiration port 162 as described above for inner tubular members 14 and 44. The aspiration port 162 communicates with the lumen 165 of the flexible inner tubular member 144 which defines an aspiration passage through the inner member 144. The cutting configuration 160 can have various configurations to cut various types of anatomical tissue with various types of cutting actions including resecting, end cutting, side cutting, shaving, burring and abrading. The aspiration port 162 can be circumscribed by the cutting configuration 160, or the aspiration port 162 can be separate and distinct from the cutting configuration. The aspiration port 162 can have various configurations and can be disposed at various locations to provide communication between a cutting site and the lumen or aspiration passage 165 of the inner tubular member 144.

The flexible inner tubular member 144 has one or more flexible regions 166 for being disposed within the one or more bends 143 of the outer member 142 to allow the inner tubular member 144 to conform to the angled configuration of the angled outer tubular member 142 while being rotatable within the angled outer tubular member. The flexible inner tubular member 144 has a plurality of flexible regions 166 and, in particular, comprises a proximal flexible region 166a and a distal flexible region 166b spaced longitudinally from the proximal flexible region 166a along the length of the inner tubular member 144 in correspondence with the proximal and distal bends 143a and 143b, respectively, of the angled outer tubular member 142. The flexible regions 166a and 166b are of sufficient length to extend within and conform to the configuration of the proximal and distal bends 143a and 143b, respectively, when the inner member 144 is rotated within the outer member 142. It should be appreciated, however, that a single flexible region 166 can be of sufficient length to extend within and conform to the configuration of a plurality of bends 143 of the outer member 142 when the inner member 144 is rotated within the outer member 142.

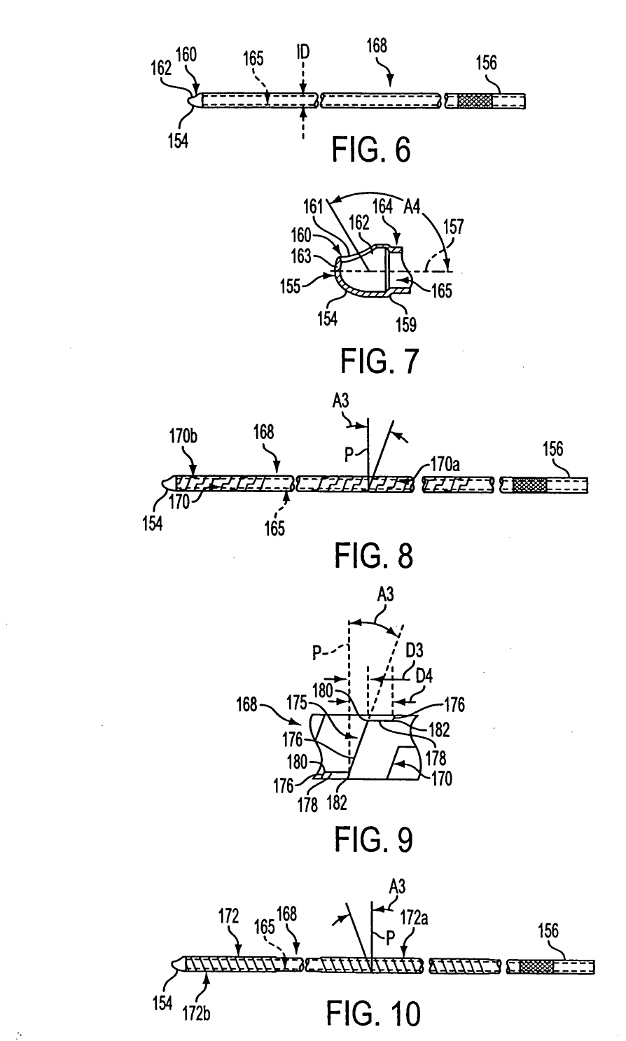

The flexible inner tubular member 144 is best depicted in FIGS. 5-10 and comprises an elongate inner tube 168, which may be made of stainless steel, having one or more continuous helical or spiral cuts 170 formed therein along one or more length portions of the inner tube 168 corresponding to the one or more flexible regions 166 to impart flexibility to the inner tube 168 along the flexible regions 166. The distal end 154 may be formed integrally, unitarily or monolithically with the inner tube 168 or as a separate component, which may be made of stainless steel, attached to a forward or distal end of the inner tube 168. An outer surface of the tube 168 may be knurled along proximal end 156 to facilitate attachment of the proximal end 156 to the inner member hub 158. The proximal end 156 may be defined by a rearward end of the inner tube 168. The lumen 165 of the flexible inner tubular member 144 is defined by the lumen 165 of the inner tube 168 and constitutes the aspiration passage of the flexible inner tubular member 144. The inner tube 168 is of unbroken solid wall construction prior to having the one or more helical cuts 170 formed therein along the one or more length portions of the inner tube 168 corresponding to the one or more flexible regions 166 as shown in FIG. 6.

FIG. 7 illustrates one possible cutting configuration 160 for the distal end 154 of the flexible inner tubular member 144. The distal end 154 comprises a cutting tip 155 joined to the forward or distal end of an elongate body of the inner tube 168 at a neck 159. The cutting tip 155 has a cutting configuration 160 including a cutting edge 161 along a peripheral edge circumscribing an opening forming aspiration port 162. The aspiration port 162 communicates with a hollow interior of the cutting tip 155 in communication with the lumen 165 of inner tube 168. The aspiration port 162 faces at an angle to the central longitudinal axis 157 of the inner tube 168, with the center of the aspiration port 162 being offset an angle A4 of about 120 degrees from the central longitudinal axis 157. A distal end wall of the cutting tip 155 has an aperture 163 therethrough communicating with the interior of the cutting tip and forming an additional aspiration port. The aperture 163 is at an angle to the central longitudinal axis 157. The cutting edge 161 includes cutting edge segments on opposite sides of the central longitudinal axis 157, and the cutting edge segments curve upwardly from the distal end wall in the proximal direction. The cutting port 152 in the outer tubular member may be circumscribed by a peripheral edge which is also a cutting edge to cooperate with the cutting edge 161.

The one or more helical cuts 170 are formed through the wall thickness of the inner tube 168 and follow a helix angle A3 in a first direction, i.e. clockwise (right hand) or counterclockwise (left hand), about the central longitudinal axis 157 of the inner tube 168. The inner tube 168 has a plurality of continuous helical cuts 170 formed therein at longitudinally spaced length portions of the inner tube 168, the inner tube 168 having a continuous proximal helical cut 170a and a continuous distal helical cut 170b spaced longitudinally from the proximal helical cut 170a by an uncut length segment of the inner tube 168 as seen in FIG. 8. The proximal and distal helical cuts 170a and 170b extend along the proximal and distal flexible regions 166, respectively, which are located in correspondence to the proximal and distal bends 143a and 143b, respectively, in the outer tubular member 142. Each helical cut 170a and 170b is continuous from end to end along the corresponding length portions, respectively, to impart flexibility to the inner tube 168 along the corresponding flexible regions 166a and 166b, respectively, while allowing the inner tube 168 to be rotated within the outer member 142. Of course, it should be appreciated that a single continuous helical cut 170 may be formed in the inner tube 168 of sufficient length to impart flexibility to the inner tube along both bends 143a and 143b in the outer member 142.

As shown in FIGS. 8 and 9, each helical cut 170 in the inner tube 168 is formed in a stepped pattern comprising repeating interconnected steps 175. Each step 175 includes a transverse or circumferential cut segment 176 extending at the helix angle A3 transverse to the length of the inner tube 168 in the first direction about the inner tube 168, and a longitudinal cut segment 178 which extends along the length of the inner tube 168 from the transverse cut segment 176 to the transverse cut segment of the next step. The transverse cut segment 176 and the longitudinal cut segment 178 of the step 175 meet at an outside corner 180 to form a step configuration. The longitudinal cut segment 178 extends from the transverse cut segment 176 at the outside corner 180 to meet the transverse cut segment of the next step at an inside corner 182. The transverse cut segment 176 meets the longitudinal cut segment of the previous step at the previous inside corner. Each helical cut 170 may be of uniform pitch along its corresponding length portion as shown for helical cuts 170a and 170b. However, a helical cut 170 can be of non-uniform pitch along its length portion to vary the flexibility of the inner tube 168 along the length portion.

Each longitudinal cut segment 178 defines the helix angle A3 with a plane P perpendicular to the central longitudinal axis 157 of the inner tube 168. The longitudinal cut segments 178 are shorter in length than the transverse cut segments 176, and the longitudinal cut segments 178 may be parallel to the central longitudinal axis 157 of the inner tube 168. The steps 175 repeat at about 100 degree rotational intervals about the central longitudinal axis 157 of the inner tube 168, with the outside corner 180 rotationally offset about 100 degrees about axis 157 from the inside corner 182 of the previous step. In one preferred embodiment, the helix angle A3 is about 20 degrees in a left hand first direction; the outside corner 180 is offset a distance D3 of about 0.005 inch from plane P in a direction perpendicular to plane P; and the inside corner 182 is offset a distance D4 of about 0.020 inch from plane P in a direction perpendicular to plane P. Preferably, each helical cut 170 is formed in the inner tube 168 by laser cutting. The distal helical cut 170b may extend all the way to the cutting tip 155 to impart flexibility to the inner tube 168 directly adjacent the cutting tip. Each helical cut 170 extends entirely through the wall thickness of inner tube 168 to impart flexibility while the inner tube 168 remains materially and structurally interconnected.

The flexible inner tubular member 144 further includes one or more single spiral or helical wrap layers 172 disposed over the helically cut length portions of inner tube 168 as shown in FIG. 10. The inner tubular member 144 includes proximal and distal single spiral wrap layers 172a and 172b disposed over the entire length of the proximal and distal helical cuts 170a and 170b, respectively. Each spiral wrap layer 172a and 172b comprises no more than a single layer of spiral or helical wrap formed by wrapping or winding a continuous strip of material helically or spirally over the helically cut length portion of inner tube 168 in a second direction, opposite the first direction of the helical cut 170, and at angle A3 with a plane P perpendicular to the central longitudinal axis 157 of the inner tube 168. Accordingly, the strips of material of the single spiral wrap layers 172a and 172b are wound around the inner tube 168 in a right hand second direction at an angle A3 of about 20 degrees with plane P. The angle A3 of the spiral wraps is the same as the angle A3 of the helical cuts but in the opposite direction from the angle of the helical cuts. Opposing ends of each strip of material are secured to the inner tube 168 such as by laser welding. Each strip of material may be about 0.050 inch in width and about 0.003 inch in thickness made from 302 stainless steel. Each flexible region 166 thusly comprises a helically cut length portion of the inner tube 168 and only a single spiral wrap layer 172 disposed over and covering the helically cut length portion.

The outer diameter of the flexible inner tubular member 144 comprises the outer diameter of the inner tube 168 and the additional thickness added by the one or more single spiral wrap layers 172 over the inner tube 168. The outer diameter of the flexible inner tubular member 144 is capable of being rotatably received in the outer tubular member 142 having the same outer diameter OD as the outer members 12 and 42. Accordingly, the flexible inner tubular member 144 may be used in combination with outer member 142 to obtain angled tissue cutting instrument 140 of the same diametric size as the straight tissue cutting instrument 10 and the angled tissue cutting instrument 40. However, the inner diameter ID of the flexible inner tubular member 144, as defined by the inner diameter ID of inner tube 168, is larger than the inner diameter ID′ of the flexible inner tubular member 44 of angled tissue cutting instrument 40 of the same diametric size as instrument 140. Also, the inner diameter ID of the flexible inner tubular member 144 is the same size as the inner diameter ID of the straight inner tubular member 14 of the straight tissue cutting instrument 10 of the same diametric size as the angled tissue cutting instrument 140. Even though the inner tubular member 144 has a spiral wrap layer 172 over the inner tube 168, the inner tubular member 144 can still be accommodated within the outer tubular member 142 of the same outer diameter as the outer tubular member 12. If necessary, clearances and/or dimensions can be adjusted within normal design tolerances while retaining sufficient strength for the inner and outer members 144 and 142. On the other hand, the outer tubular member 42 of the same size angled tissue cutting instrument 40 cannot accommodate a flexible inner tubular member 44 in which the outer and inner spiral wrap layers 74 and 72 are disposed over an inner tube 68 having inner diameter ID. Rather, a flexible inner tubular member 44 in which the outer and inner spiral wrap layers 74 and 72 are disposed over an inner tube 68 having inner diameter ID will require an outer tubular member 42 of larger outer diameter than outer diameter OD such that an inner tube 68 having an inner diameter ID must be used in a larger size instrument 40 compared to the sizes of instruments 10 and 140 in which an inner member having inner diameter ID is utilized. With the present invention, the inner tube 68 of inner tubular member 44 could be used as the inner tube 168 of an angled tissue cutting instrument 140 of smaller size than the instrument 40, and an inner tube the same as the inner tube 14 of straight instrument 10 can be used as the inner tube 168 in an angled instrument of the same diametric size as the straight instrument.

Operation of angled tissue cutting instrument 140 to cut anatomical tissue in a cutting procedure is similar to that described above for instruments 12 and 40. As the flexible inner tubular member 144 is rotated within the angled outer tubular member 142, the one or more flexible regions 166 transmit torque to the cutting configuration 160. The one or more helical cuts 170, being left hand, will tighten as the inner member 144 is rotated in a forward rotational direction. The stepped pattern of the one or more helical cuts 170 assists in transmitting torque, and the one or more right hand spiral wrap layers 172 tighten down onto the inner tube 168 when the flexible inner tubular member 144 is rotated in a reverse rotational direction. Accordingly, the angled tissue cutting instrument 140 may be operated to apply torque in both forward and reverse rotational directions while minimizing wind-up. Materials at the cutting site are aspirated from the patient's body through the flexible inner tubular member 144 via the aspiration ports 162, 163 and the aspiration passage 165 when suction or vacuum is produced in the aspiration passage. Aspiration efficiency is increased and the risk of clogging is reduced with the angled tissue cutting instrument 140 in comparison to an angled tissue cutting instrument 40 of the same size due to the greater diametric size of the aspiration passage 165 defined by the inner diameter ID of the flexible inner tubular member 144. Tissue cutting is thusly accomplished with the angled tissue cutting instrument 140 in essentially the same manner as a counterpart straight tissue cutting instrument 10 of the same size as the angled tissue cutting instrument 140. Irrigating fluid may be supplied via the connector 164 to the lumen of the outer tubular member 142 for discharge at the cutting site through cutting port 152, the irrigating fluid flowing between the inner diameter of the outer tubular member 142 and the outer diameter of the inner tubular member 144.

The angled tissue cutting instrument 140 may be made available in various standard diametric sizes including 2.9 mm, 3.5 mm and 4.0 mm. For a given size angled tissue cutting instrument 140, an inner tube the same size as the inner tube 14 of a smaller size straight instrument 10 does not have to be employed as the inner tube 168. Rather, standard sizes of angled tissue cutting instruments 140 can be constructed using inner tubes 168 having the same inner diameters ID as the inner tubes 14 of straight tissue cutting instruments 10 of the corresponding size. Accordingly, the inner tubular members of straight and angled tissue cutting instruments of the same size can be fabricated using the same size inner tubes for the same or essentially the same aspiration efficiency and risk of clogging.

FIGS. 5-10 also depict a method of fabricating an angled tissue cutting instrument 140 according to the present invention and, in particular, a method of fabricating a flexible inner tubular member 144 of an angled tissue cutting instrument. The method involves forming a continuous helical cut 170 along a length portion of the elongate inner tube 168 at the angle A3 in the first direction about the inner tube to impart flexibility along the length portion of the inner tube. The length portion of the inner tube 168 along which the helical cut 170 is formed corresponds to a flexible region 166 to be disposed within a bend 143 of the angled outer tubular member 142. Prior to having the helical cut 170 formed therein, the inner tube 168 is of solid wall construction, at least along the designated length portion. Also, the inner tube 168 has the inner diameter ID which is the same size as the inner diameter ID of the elongate inner tube forming the straight inner tubular member 14 of the straight tissue cutting instrument 10 of the same diametric size as the angled tissue cutting instrument 140. Once the helical cut 170 is formed in the inner tube 168, preferably by laser cutting, the continuous strip of material used for the spiral wrap layer 172 is spirally wound over the helically cut length portion of the inner tube 168 at the angle A3 but in the second direction about the inner tube, opposite the first direction of the helical cut 170, to form no more than a single spiral wrap layer over the inner tube. Opposing ends of the strip of material are secured to the inner tube, such as by welding, to form a flexible region 166 along the length portion of the inner tube. The flexible inner tubular member 144 is inserted within the angled outer tubular member 142 with the flexible region 166 disposed within the bend 143 in the outer tubular member and the cutting configuration 160 of the inner tubular member exposed by the cutting port 152 in the distal end of the outer tubular member. The inner tubular member 144 is rotatably disposed in the outer tubular member 142, and the flexible region 166 transmits torque to rotate the cutting configuration 160 when the inner tubular member 144 is rotated relative to and within the outer tubular member 142. The outer tubular member 142 within which the inner tubular member 144 is disposed has an outer diameter the same size as the outer diameter of the straight outer tubular member 12 of the straight tissue cutting instrument 10 of the same diametric size as the angled tissue cutting instrument 140.

Inasmuch as the present invention is subject to many variations, modifications and changes in detail, it is intended that all subject matter discussed above or shown in the accompanying drawings be interpreted as illustrative only and not be taken in a limiting sense.

1. An angled tissue cutting instrument comprising
an elongate angled outer tubular member having a proximal end, a distal end, a bend between said proximal end and said distal end, and an opening at said distal end defining a cutting port; and
an elongate flexible inner tubular member rotatably disposed within said outer tubular member to transmit torque in forward and reverse rotational directions, said inner tubular member comprising an elongate inner tube of solid wall construction having a proximal end and a distal end, a continuous helical cut formed in a stepped pattern along a length portion of said inner tube corresponding to said bend, said helical cut being formed in said inner tube at an angle in a first direction about said inner tube to impart flexibility by which said inner tube conforms to said angled outer tubular member while being rotated within said angled outer tubular member, a cutting configuration carried at said distal end of said inner tube for exposure by said cutting port to cut anatomical tissue when said inner tubular member is rotated within said outer tubular member, and no more than a single layer of spiral wrap disposed over said helical cut, said single layer of spiral wrap extending along said length portion at said angle in a second direction, opposite said first direction, about said inner tube.

2. The angled tissue cutting instrument recited in claim 1 wherein said outer tubular member is angled at a plurality of bends spaced along the length of said outer tubular member.

3. The angled tissue cutting instrument recited in claim 2 wherein said flexible inner tubular member comprises a plurality of said helical cuts formed in said inner tube along a plurality of said length portions in correspondence with said plurality of bends, respectively, and a plurality of said single layers of spiral wrap disposed over said plurality of helical cuts, respectively, with said plurality of said single layers of spiral wrap extending along said plurality of said length portions, respectively.

4. The angled tissue cutting instrument recited in claim 3 wherein said plurality of bends extend in different directions and at different angles.

5. The angled tissue cutting instrument recited in claim 4 wherein said outer tubular member includes a straight proximal length portion extending distally from said proximal end of said outer tubular member to a proximal bend, a straight intermediate length portion extending distally from said proximal bend to a distal bend, said proximal bend being bent in a first direction at an angle of about 45 degrees to a central longitudinal axis of said proximal length portion, said distal bend being bent in a second direction opposite said first direction of said proximal bend, at an angle of about 15 degrees to a central longitudinal axis of said intermediate length portion.

6. The angled tissue cutting instrument recited in claim 1 wherein said single layer of spiral wrap comprises a continuous strip of material spirally wound over said inner tube.

7. The angled tissue cutting instrument recited in claim 1 wherein said helical cut is formed in a stepped pattern comprising repeating interconnected steps.

8. The angled tissue cutting instrument recited in claim 7 wherein said steps repeat at rotational intervals of about 100 degrees about said inner tube.

9. An angled tissue cutting instrument comprising
an elongate angled outer tubular member having a proximal end, a distal end, a bend between said proximal end and said distal end, and an opening at said distal end defining a cutting port; and
an elongate flexible inner tubular member rotatably disposed within said outer tubular member to transmit torque in forward and reverse rotational directions, said inner tubular member comprising an elongate inner tube of solid wall construction having a distal end, a proximal end, a central longitudinal axis, an aspiration passage through said inner tube, a continuous helical cut formed in a stepped pattern along a length portion of said inner tube in correspondence with said bend to impart flexibility by which said inner tube conforms to said angled outer tubular member while being rotated within said outer tubular member, a cutting configuration carried by said distal end of said inner tube for exposure by said cutting port to cut anatomical tissue when said inner tube is rotated within said outer tubular member, an aspiration port at said distal end of said inner tube establishing communication with said aspiration passage, and no more than one layer of spiral wrap wound over said length portion, said stepped pattern comprising repeating interconnected steps each made up of a transverse cut segment extending transverse to the length of said inner tube in a first direction about said inner tube and at an angle to a plane perpendicular to said central longitudinal axis, and a longitudinal cut segment extending from said transverse cut segment along the length of said inner tube, said spiral wrap being wound over said length portion in a second direction, opposite said first direction, about said inner tube and at said angle to a plane perpendicular to said central longitudinal axis.

10. The angled tissue cutting instrument recited in claim 9 wherein said angle is about 20 degrees.

11. The angled tissue cutting instrument recited in claim 9 wherein said first direction is a left hand direction about said central longitudinal axis and said second direction is a right hand direction about said central longitudinal axis.

12. The angled tissue cutting instrument recited in claim 9 wherein said steps repeat at rotational intervals of about 100 degrees about said central longitudinal axis.

13. The angled tissue cutting instrument recited in claim 9 wherein said longitudinal cut segment has a length along said central longitudinal axis and said transverse cut segment has a length transverse to said central longitudinal axis greater than said length of said longitudinal cut segment.

14. A method of fabricating an angled tissue cutting instrument comprising
forming a continuous helical cut along a length portion of an elongate inner tube at an angle in a first direction about said inner tube to impart flexibility along the length portion, the inner tube being of solid wall construction prior to having the helical cut formed therein and having an inner diameter the same size as the inner diameter of an elongate inner tube forming the inner tubular member of a straight tissue cutting instrument of the same diametric size as the angled tissue cutting instrument;
wrapping a continuous strip of material spirally over the helically cut length portion of the inner tube at the angle in a second direction, opposite the first direction, about the inner tube to form no more than a single layer of spiral wrap over the inner tube;
securing opposing ends of the strip of material to the inner tube to form a flexible inner tubular member having a flexible region along the length portion of the inner tube; and
inserting the flexible inner tubular member for rotation within an angled outer tubular member with the flexible region disposed within a bend in the outer tubular member and a cutting configuration of the flexible inner tubular member exposed by a cutting port in a distal end of the outer tubular member, the outer tubular member having an outer diameter the same size as the outer diameter of a straight outer tubular member of the straight tissue cutting instrument of the same diametric size as the angled tissue cutting instrument.

15. The method recited in claim 14 wherein said step of forming includes forming the helical cut in an elongate inner tube which may also be used as the inner tubular member of the straight tissue cutting instrument of the same diametric size as the angled tissue cutting instrument.

16. The method recited in claim 14 wherein said step of forming includes forming the helical cut in a stepped pattern comprising repeating interconnected steps.

17. The method recited in claim 14 wherein said step of forming includes forming the helical cut in an inner tube of a 2.9 mm angled tissue cutting instrument using an inner tubular member having an inner diameter the same size as the inner diameter of an inner tubular member of a 2.9 mm straight tissue cutting instrument.

18. The method recited in claim 14 wherein said step of forming includes forming the helical cut in an inner tube of a 3.5 mm angled tissue cutting instrument using an inner tube having an inner diameter the same size as the inner diameter of an inner tubular member of a 3.5 mm straight tissue cutting instrument.

19. The method recited in claim 14 wherein said step of forming includes forming the helical cut in an inner tube of a 4.0 mm angled tissue cutting instrument using an inner tube having an inner diameter the same diametric size as the inner diameter of an inner tubular member of a 4.0 mm straight tissue cutting instrument.

20. The method recited in claim 14 wherein said step of forming the helical cut includes forming the helical cut at an angle of about 20 degrees in a left hand direction and said step of wrapping includes wrapping the strip of material at an angle of about 20 degrees in a right hand direction.

21. The method recited in claim 14 wherein said step of inserting includes providing an annular clearance between the outer diameter of the flexible inner tubular member and the inner diameter of the outer tubular member for the flow of irrigating fluid along the angled tissue cutting instrument.

22. The method recited in claim 16 wherein said step of forming includes forming the steps at rotational intervals of about 100 degrees about a central longitudinal axis of the inner tube.
